(19) United States
(12) Patent Application Publication
(10) Pub. No.: US 2013/0171072 A1
(43) Pub. Date: Jul. 4, 2013

(54) DYES AND METHODS OF SELECTING DYES FOR USE IN PERSONAL CARE PRODUCTS

(71) Applicant: Catherine Schmit, Glendale, AZ (US)

(72) Inventor: Catherine Schmit, Glendale, AZ (US)

(73) Assignee: THE DIAL CORPORATION, Scottsdale, AZ (US)

(21) Appl. No.: 13/339,078

(22) Filed: Dec. 28, 2011

(51) Int. Cl. A61K 49/00; A61Q 90/00; A61Q 15/00; G01N 21/00; A61K 8/40

(52) U.S. Cl. 424/9.2; 436/164; 424/63

(57) ABSTRACT

Dyes and methods of selecting dyes for use in personal care products are provided. An exemplary method includes selecting a dye concentration of a dye that is brightly or intensely colored, determining a dye concentration of the dye that does not change the color of skin or minimally changes the color of skin, and determining a dye concentration of the dye that does not transfer color or minimally transfers color. An exemplary dye includes, by weight percent of the personal care product in which such dye is provided, approximately 0.001500% Red 33 dye and 0.003750% Orange 4 dye.

### TECHNICAL FIELD

The present disclosure relates generally to dyes for use in personal care products and methods of selecting dyes for use in personal care products. More specifically, the present disclosure relates to dyes and methods of selecting dyes exhibiting improved brightness, without color transfer or staining characteristics.

### BACKGROUND

Colorants are used to provide permanent, semi-permanent, and temporary color to personal care products for the hair and skin. Often, the colorants used are dyes, which can cause staining or coloring on the user's skin, the user's clothing, or other fabrics with which the user comes in contact. In many applications, change of skin color perception is undesirable, which has heretofore limited the use of dyes in personal care products.

For aesthetic reasons, for example, it is desirable to provide personal care products with bright and/or intense coloring. However, in order to provide bright and/or intense coloring, the use of dyes may be required, which could result in a personal product that leaves undesirable change in skin color perception upon use. As such, careful selection of dyes is required in order to avoid or minimize possible change in skin color perception.

Currently, colored personal care products are tested after development in ensure that they do not have negative attributes, for example, change in skin color perception. This may require a number of different dye systems to be developed as “back-ups” in case the primary dye system is found to have negative attributes. This trial-and-error method of dye selection may result in a significant amount of time, resources, and money wasted before a suitable color system is found.

Accordingly, it is desirable to provide dyes for use in personal care products that minimize or eliminate change in color perception of skin and/or staining of clothing. In addition, it is desirable to provide methods for the selection of dyes for use in personal care products that are more efficient than the trial-and-error approaches known currently in the art. Furthermore, other desirable features and characteristics will become apparent from the subsequent detailed description and the appended claims, taken in conjunction with the accompanying drawings and this background.

### BRIEF SUMMARY

Dyes and methods of selecting dyes for use in personal care products are provided. In one embodiment, an exemplary method includes selecting a dye concentration of a dye that is brightly or intensely colored, determining a dye concentration of the dye that does not change the color of skin or minimally changes the color of skin, and determining a dye concentration of the dye that does not transfer color or minimally transfers color to consumers' clothing.

In another embodiment, an exemplary dye includes, by weight percent of the personal care product in which such dye is provided, approximately 0.001500% Red 33 dye and 0.003750% Orange 4 dye.

In yet another embodiment, an exemplary method includes selecting a dye concentration that is brightly or intensely colored by testing a series of dye concentrations using a spectroscopic analysis and determining from the series of dye concentrations a dye concentration that does not change the color of skin or minimally changes the color of skin by applying a personal care product to the skin and visually inspecting the skin. The method further includes determining from the series of dye concentrations a dye concentration that does not transfer color or minimally transfers color by applying the personal care product to the skin, contacting the skin with a color transfer substrate, and visually inspecting the color transfer substrate, and determining from the series of dye concentrations a dye concentration that does not transfer color or minimally transfers color by applying the personal care product to a staining substrate, washing, and visually inspecting the staining substrate before and after washing.

### BRIEF DESCRIPTION OF THE DRAWINGS

The present invention will hereinafter be described in conjunction with the following drawing figures, wherein like numerals denote like elements, and wherein:

FIG. 1 is block diagram illustrating an exemplary method for selecting a dye for use it a personal care product; and

FIG. 2 is a dimensionless graphical representation of example dye concentration ranges determined in accordance with the methods presented herein.

### DETAILED DESCRIPTIONS

The following detailed description is merely exemplary in nature and is not intended to limit the invention or the application and uses of the invention. Furthermore, there is no intention to be bound by any theory presented in the preceding background of this disclosure or the following detailed description.

The various embodiments contemplated herein include dyes for use in personal care products and methods of selecting dyes for use in personal care product. Personal care products can include, but are not limited to, lotions, creams, gels, shampoos, hair coloring agents, skin treatment agents, oral care compounds, deodorants, and antiperspirant, among others. The dyes disclosed herein, when used in personal care products, result in a desirably brightly and/or intensely colored product that reduces, minimizes, or eliminates the incidence of change in color perception to the user's skin, the user's clothing, or other fabrics or items with which the user comes in contact. The methods disclosed herein beneficially predetermine useful dye systems that, when used in personal care products, result in a desirably brightly and/or intensely colored product.

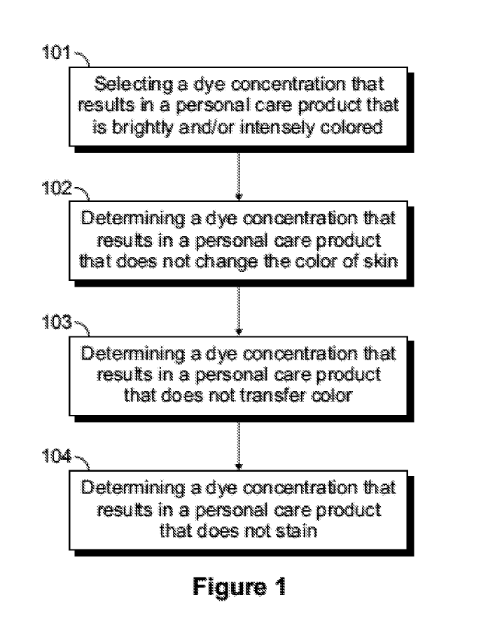

In one embodiment, as shown in FIG. 1, a method for the selection of dyes for use in personal care products includes determining a dye concentration (i.e., a quantity of dye used per unit volume of personal care product) that, when used in a personal care product will be considered brightly and/or intensely colored (101). To make this determination, a dye or series of dyes is first selected for testing based on, for example, the aesthetic qualities of the dye or series of dyes. Each dye is made into a dye solution or solutions (hereinafter “solutions”) with a known percent of active dye contained therein. The various dye solutions are then tested using an instrument for performing a spectroscopic or spectrophotometric analysis on the dye solutions. While any form of spectroscopic or spectrophotometric analysis may be employed, an exemplary embodiment includes the use of an instrument for performing an absorbance spectra, for example, an absorption spectrophotometer. The testing results in the determination of at least one empirical spectroscopic or spectrophotometric characteristic with respect to the series of dye solutions. In the example of an absorbance spectra, a wavelength maxima can be determined for each dye solution in the series.

The dye solutions are then visually inspected to determine which dye solutions have the desired color brightness or intensity characteristics. This determination is subjective in nature, and is based on the personal care product manufacturers' subjective determination as to what visual qualities are desirable based on aesthetic considerations. Once dye solutions are visually inspected, a correlation can be made between the spectroscopic instrument testing results and the dyes determined to have the requisite brightness and/or intensity characteristics by visual inspection. In the example of an absorbance spectra brightly and/or intensely colored dyes will have absorbances that correlate with the minimum dye solutions determined to be subjectively bright and/or intensely colored. Dye solutions below the minimum level may be removed from further consideration.

Table 1, set forth below, presents the result of a spectrophotometric analysis of several commercially available drug and cosmetic (D & C) and food, drag, and cosmetic (FD & C) dyes, diluted at an arbitrary level for analysis:

TABLE 1

| | | | |
|---|---|---|---|
| | Cl | | Min AU |
| Dye | Number | Wavelength | Absorbance |

Yellow Hue

| | | | |
|---|---|---|---|
| FD&C Yellow 5 | 19140 | 426 | 0.90 |
| D&C Yellow 8 | 45350 | 490 | 1.00 |
| D&C Yellow 10 | N/A | 410 | 0.60 |

Orange Hue

| | | | |
|---|---|---|---|
| FD&C Yellow 5 | 15985 | 485 | 2.00 |
| D&C Orange 4 | 15510 | 485 | 2.00 |

Red Hue

| | | | |
|---|---|---|---|
| FD&C Red 4 | 14700 | 505 | 1.00 |
| FD&C Red 40 | 16035 | 505 | 1.40 |
| D&C Red 33 | 17200 | 527 | 1.00 |

Blue Hue

| | | | |
|---|---|---|---|
| FD&C Blue 1 | 42090 | 630 | 1.50 |
| FD&C Green 3 | 42053 | 623 | 1.00 |

wherein Cl is the Color Index number, wavelength is measured in nanometers, and the minimum absorbance is measured in Absorbance Units (AU). The absorbances determined in Table 1 can be increased by decreasing the dilution of the dyes, and decreased by increasing the dilution of the dyes.

Based on the empirical spectroscopic determination of dye color brightness and/or intensity, a series of test personal care products can be produced having increasing quantities of dye contained therein. For example, a first test product may include the minimum level of dye empirically determined to be bright and/or intensely colored, and additional test products can have sequentially increasing amounts of dye contained therein. As such, a series of test personal care products is produced for further testing.

A method for the selection of dyes for use in personal care products may further include determining a maximum concentration of dye in a personal care product above which skin color is perceived as changed when such personal care product is applied to the skin in the prescribed manner (102). To make this determination, the series of test personal care products each can be applied to the skin. After application of each such test product, a determination is made whether the skin has perceptibly changed color. This determination is subjective, and is based on the visual qualities of the skin before and after application of the test product.

Product application may be done using a controlled testing protocol. The protocol can be determined depending on the type of personal care product tested. In the example of a deodorant compound, for example a deodorant stick or the like, a suitable protocol may include application of a test stick to the arm using a set number of strokes in prescribed directions, for example, five up strokes and five down strokes for a total of ten strokes. Thereafter, the skin is inspected. If a change in color is perceived, then the test product is determined to have an undesirable high concentration of dye. As such, with regard to the series of test products previously produced, a maximum suitable level of dye concentration may be determined through visual inspection of the skin. Dyes having a concentration greater than the maximum level may be removed from further consideration.

A method for the selection of dyes for use in personal care products may further include determining a maximum dye level that does not result in color transfer (103). As discussed above, color transfer is undesirable to, for example, the user's clothing or other fabrics or items with which the user comes in contact. To make this determination, the test personal care products can be each applied to the skin. After application of each such test product, a color transfer substrate, which may include a white or light colored fabric, paper towel, or other medium, may be contacted with the skin, for example, wiped across the skin. The fabric, paper towel, or other medium is visually inspected for the transfer of color. This determination is subjective, and is based on the visual qualities of the test fabric before and after contact with the skin.

Product application may be done using a controlled testing protocol. The protocol can be determined depending on the type of personal care product tasted. In the example of a deodorant stick or the like, a suitable protocol may include application of a test stick to the arm using a set number of stroke in prescribed directions, for example, five up strokes and five down strokes for a total of ten strokes. A suitable protocol may further include contacting the skin with a fabric or other medium such as a paper towel in a prescribed manner. The fabric or paper towel can be white in color so that any color transfer is readily detectible by visual inspection. The prescribed manner of contact may include wiping the skin with a given level of pressure for a set number of times. For example, the skin may be wiped five times with a paper towel or fabric using five pounds of pressure. If a change in color is perceived on the fabric or paper towel, then the test product is determined to have an undesirable high concentration of dye. As such, with regard to the series of test products previously produced, a maximum suitable level of dye concentration with regard to color transfer may be determined through visual inspection. Dyes having a concentration greater than the maximum level may be removed from further consideration.

A method for the selection of dyes for use in personal care products may further include determining a maximum concentration of dye in a personal care product above which a fabric is stained when such personal care product is directly applied to the fabric (104). The series of test personal care products each can be applied to a test staining substrate, such as fabric. After application of each such test product, a determination is made whether the fabric has perceptibly stained, for example, perceptibly changed color after washing. This determination is subjective, and is based on the visual qualities of the fabric before and after application of the test product and washing.

Product application may be done using a controlled testing protocol. The protocol can be determined depending on the type of personal care product tested. In the example of a deodorant stick or the like, a suitable protocol may include application of a test stick to the fabric using a set number of strokes in prescribed directions, for example, five up strokes and five down strokes for a total of ten strokes. Thereafter, the fabric is inspected before and after washing. If a change in color is perceived after washing, then the test product is determined to have an undesirable high concentration of dye. As such, with regard to the series of test products previously produced, a maximum suitable level of dye concentration may be determined through visual inspection. Dyes having a concentration greater than the maximum level may be removed from further consideration.

It will be appreciated based on the foregoing description that a dye system that is bright and/or intensely colored, does not change the color of skin, does not transfer color, and does not stain will be suitable for use in leave-on personal care products according to the present disclosure, and may be selected therefor. However, it will also be appreciated that a suitable dye system need not completely achieve the aforementioned desirable characteristics to be suitable. For example, in some instances, a dye system that changes the color of skin minimally might be also acceptable. A dye system that transfers only minimal color might be acceptable. Further, a dye system that results in minimal staining might be acceptable. As each such characteristic is necessarily subjective, persons skilled in the art will be able to make determinations accordingly to find suitable dye systems in accordance with the methods presented herein.

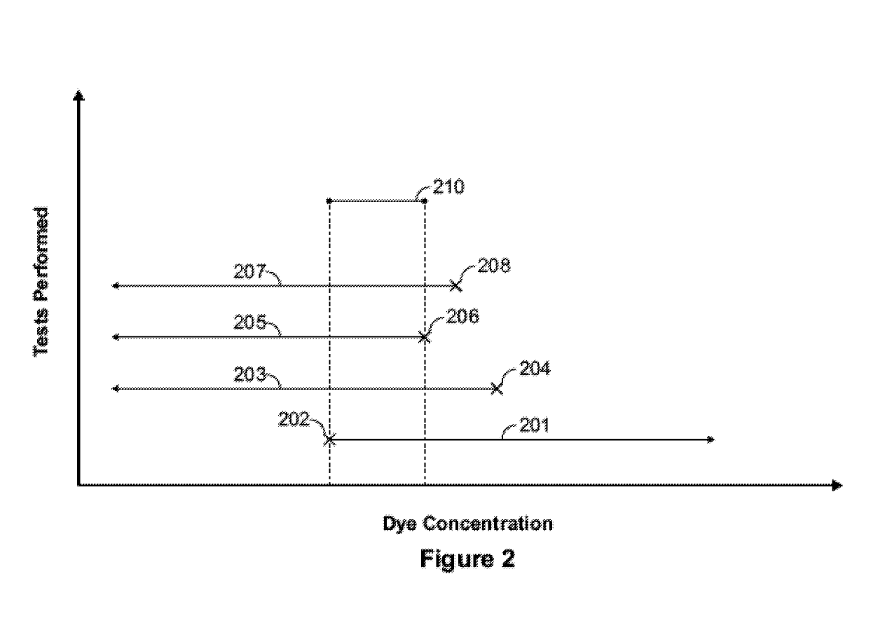

FIG. 2 presents a dimensionless graphical representation of exemplary dye concentration ranges that may be determined in accordance with the methods presented herein. As shown in FIG 2, a suitable color brightness range 201 includes a minimum concentration 202 for achieving the desired brightness, with increasing concentrations above the minimum included within the range 201. A suitable range 203 wherein skin color is not perceived (or only minimally perceived) to change includes a maximum concentration 204 for preventing skin color change, with decreasing concentrations below the maximum level included within the range. A suitable range 205 wherein color is not perceived (or only minimally perceived) to transfer from the skin includes a maximum concentration 206 for preventing color transfer, with decreasing concentrations below the maximum level included within the range. A suitable range 207 wherein fabric is not perceived (or only minimally perceived) to stain after direct application includes a maximum concentration 208 for preventing staining, with decreasing concentrations below the maximum level included within the range. The overlapping range 210 of ranges 201,203,205,207 depicts a range of dye concentrations having the desired color brightness and/or intensity characteristics, no or minimal perceived skin color change, no or minimal perceived color transfer, and no or minimal perceived staining. As such, the overlap range 210 depicts a suitable range of dye concentrations from which to select for use in leave-on personal care products in accordance with the present disclosure.

While the methods heretofore described include determinations with regard to brightness and or intensity of color, skin color change, color transfer, and with regard to direct application staining, methods in accordance with the present disclosure may include additional considerations for the selection of desirable dyes. For example, even if a dye is determined to be suitable based on the above testing, the dye may be removed from consideration for other reasons such as cost of production, difficultly in manufacturing, availability of the dye, and other reasons that will be appreciated by those having ordinary skill in the art.

Once a dye or series of dyes has been selected according to the procedures described above, suitable personal care products can be developed by combining such dye or dyes with such personal care products. As will be appreciated, dyes can be combined in a variety of formulations to create a variety of colors, brightness of colors, and/or intensity of colors to suit particular aesthetic requirements for the personal care products.

### EXAMPLE

The following is an exemplary embodiment of a dye-based color system selected in accordance with the methods of the present disclosure. The example is provided for illustration purposes only and is not meant to limit the various embodiments of dyes or methods of selecting dyes for use in personal care products. All materials are set forth in weight percent of the personal care product.

Three color systems were selected as having desirable aesthetic qualities for possible use in personal care products. The color systems, hereinafter referred to as Color System 1, Color System 2, and Color System 3, were created using mixtures of the following dyes: Red 40, Red 33, Orange 4, and Blue 1. The color systems were created according to the following formulae, wherein the percentages provided are approximate, including a +/−15% variance in stated concentration:

| Dye % | Color System 1 | Color System 2 | Color System 3 |
|---|---|---|---|
| Red 40 | 0.030000 | 0 | 0.010000 |
| Red 33 | 0 | 0.001500 | 0 |
| Orange 4 | 0.000600 | 0.003750 | 0.006000 |
| Blue 1 | 0.000010 | 0 | 0 |

Color Systems 1 through 3 were selected from a larger population of color systems for testing based on their color brightness and intensity characteristics. It was determined that Color Systems 1 through 3 had the best color, brightness, and intensity characteristic of the larger population, based on a subjective inspection of each member of the population. As discussed above, a minimum concentration of a particular dye in solution to achieve the desired brightness and/or intensity characteristic can be determined through the use of spectroscopic instruments. The active dye component of the solution can then be incorporated into a personal care product for testing. Color Systems 1 through 3 were each included in a deodorant stick for producing a series of three test personal care products. The deodorant sticks had the same formula except for the dye incorporated therein.

In a first test to determine skin color change, three test deodorant sticks each including a respective one of Color Systems 1 through 3 were applied to human skin. Each stick was applied using five up strokes and five down strokes for a total of ten strokes each. The skin was visually inspected. Color System 1 resulted in a pronounced red appearance on the skin. Color System 2 resulted in no color change on the skin. Color System 3 resulted in a slight red appearance on the skin.

In a second test to determine color transfer, the three test deodorant sticks each including a respective one of Color Systems 1 through 3 were applied to human skin. Each stick was applied using five up strokes and five down strokes for a total of ten strokes each. A white paper towel was then wiped across the skin five times, using approximately five pounds of pressure on each stroke. The paper towel was visually inspected. Color System 1 was observed to transfer a high amount of color to the paper towel. Color System 2 was observed to transfer no color to the paper towel. Color System 3 was observed to transfer a minimal amount of color to the paper towel.

In a third test to determine direct-application staining, the three test deodorant sticks each including a respective one of Color Systems 1 through 3 were applied to a white fabric, specifically a while T-shirt. Each stick was applied using five up strokes and five down strokes for a total of ten strokes each. The fabric was visually inspected. Color System 1 was observed to leave a bright stain on the fabric. Color System 2 was observed to leave a minimal stain on the fabric. Color System 3 was observed to leave a bright stain on the fabric. The T-shirts were then allowed to dry for four hours, followed by washing in a commercial washing machine with detergent. The fabric was visually inspected again. Color System 1 was observed to leave a slight stain on the fabric. Color System 2 was observed to leave no stain on the fabric. Color System 3 was observed to leave a slight stain on the fabric.

Based on the aforementioned testing, it was therefore determined that Color System 2 met the requirements of having the desired color brightness and/or intensity characteristics, no or minimal perceived skin color change, no or minimal perceived color transfer, and no or minimal perceived staining. As such, it was determined that Color System 2 was a suitable dye system for use in personal care products, and particularly in deodorant compounds, and more particularly in deodorant sticks. Color Systems 1 and 3 were not selected.

Further based on the aforementioned testing. Color System 2 was made into a dye for use in personal care products. The dye based on Color System 2 includes dyes Red 33 and Orange 4 in a relative ratio of about 15/37.5. When included in a personal care product, the dye based on Color System 2 is provided in an amount of about 0.001500 percent by weight of the personal care product. Red 33 dye and about 0.003750 percent by weight of the personal care product Orange 4 dye. Suitable personal care products include, but are not limited to, deodorants and antiperspirants, etc., as discussed above. Formulations for deodorants, antiperspirants, and other personal care products are well known to those having ordinary skill in the art. An example deodorant composition includes about 0.001500 percent by weight Red 33 dye and about 0.003750 percent by weight Orange 4 dye. An example antiperspirant composition includes about 0.001500 percent by weight Red 33 dye and about 0.003750 percent by weight Orange 4 dye.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope defined by the claims, which includes known equivalents and foreseeable equivalents at the time of filing this patent application.

What is claimed is:

1. A method for selecting a dye for use in a personal care product, comprising:
selecting a dye concentration of a dye that is brightly or intensely colored;
determining a dye concentration of the dye that does not change the color of skin or minimally changes the color of skin; and
determining a dye concentration of the dye that does not transfer color or minimally transfers color.

2. The method of claim 1, further comprising determining a dye concentration of the dye that does not slain or minimally stains.

3. The method of claim 2, wherein determining a dye concentration of the dye that does not change the color of skin or minimally changes the color of skin, determining a dye concentration of the dye that does not transfer color or minimally transfers color, or determining a dye concentration of the dye that does not stain or minimally stains comprises a subject determination based on: a visual inspection of the skin, a visual inspection of a color transfer substrate, or a visual inspection of a staining substrate, respectively.

4. The method of claim 3, wherein determining a dye concentration of the dye that does not change the color of skin or minimally changes the color of skin comprises applying a personal care product containing the dye to the skin and visually inspecting the skin for color change.

5. The method of claim 3, wherein determining a dye concentration of the dye that does not transfer color or minimally transfers color comprises applying a personal care product containing the dye to the skin, contacting the skin with the color transfer substrate, and visually inspecting the color transfer substrate.

6. The method of claim 3, wherein determining a dye concentration of the dye that does not stain or minimally stains comprises applying a personal care product containing the dye to the staining substrate and visually inspecting the staining substrate.

7. The method of claim 1, wherein determining a dye concentration of the dye that is brightly of intensely colored comprises a subjective determination based on visual inspection of the dye.

8. The method of claim 1, wherein determining a dye concentration of the dye that is brightly or intensely colored comprises an objective determination based on use of a spectrophotometric instrument.

9. The method of claim 8, wherein determining a dye concentration of the dye that is brightly or intensely colored comprises an objective determination based on an absorbance spectra.

10. The method of claim 1, wherein determining a dye concentration of the dye that does not change the color of skin or minimally changes the color of skin comprises determining a range of concentrations of the dye that do not change the color of skin or minimally change the color of skin, wherein determining a dye concentration of the dye that does not transfer color or minimally transfers color comprises determining a range of concentrations of the dye that do not transfer color or minimally transfer color, and wherein determining a dye concentration than does not stain or minimally stains comprises determining a range of concentrations of the dye that do not stain or minimally stain.

11. The method of claim 10, further comprising incorporating into a leave-on personal care product the dye within the range of concentrations that do not change the color of skin or minimally change the color of skin, within the range of concentrations that do not transfer color or minimally transfer color, and within the range of concentrations that do not stain or minimally slain.

12. The method of claim 11, further comprising incorporating into a deodorant compound the dye within the range of concentrations that do not change the color of skin or minimally change the color of skin, within the range of concentrations that do not transfer color or minimally transfer color, and within the range of concentrations that do not stain or minimally stain.

13. A dye for use in a personal care product comprising, by weight percent of the personal care product in which such dye is provided, approximately 0.001500% Red 33 dye and approximately 0.003750% Orange 4 dye.

14. The dye for use in a personal care product of claim 13, wherein the personal care product is a deodorant or an antiperspirant.

15. A method for selecting a dye for use in a personal care product, comprising:
selecting a dye concentration that is brightly or intensely colored by testing a series of dye concentrations using a spectrophotometric and visual analysis;
determining from the series of dye concentrations a dye concentration that does not change the color of skin or minimally changes the color of skin by applying a personal care product to the skin and visually inspecting the skin;
determining from the series of dye concentrations a dye concentration that does not transfer color or minimally transfers color by applying the personal care product to the skin, contacting the skin with a color transfer substrate, and visually inspecting the color transfer substrate; and
determining from the series of dye concentrations a dye concentration that does not stain or minimally stains by applying the personal care product to a staining substrate and visually inspecting the staining substrate.

16. The method of claim 15, wherein contacting the skin with a color transfer substrate comprises contacting the skin with a paper towel and wherein applying the personal care product to a staining substrate comprises applying the personal care product to a fabric.

17. The method of claim 15, wherein visually inspecting the skin, visually inspecting the color transfer substrate, and visually inspecting the staining substrate comprise a subjective visual inspection.

18. The method of claim 15, wherein determining a dye concentration of the dye that is brightly or intensely colored comprises an objective determination based on use of a spectrophotometric instrument.

19. The method of claim 18, wherein determining a dye concentration of the dye that is brightly or intensely colored comprises an objective determination based on an absorbance spectra.

20. The method of claim 19, wherein determining a dye concentration of the dye that is brightly or intensely color comprises determining a minimum absorbance of the absorbance spectra.
